(19) United States
(12) Patent Application Publication
(10) Pub. No.: US 2023/0303507 A1
(43) Pub. Date: Sep. 28, 2023

(54) RIGID CANNABIDIOL ANALOGUES AS POTENT MODULATORS OF CANNABINOID RECEPTORS AND USES THEREOF

(71) Applicant: LONDON PHARMACEUTICALS AND RESEARCH CORPORATION, London (CA)

(72) Inventor: Mahmoud Mohamed Abdrabo MOUSTAFA, London, Ontario (CA)

(21) Appl. No.: 18/007,935

(22) PCT Filed: Jun. 4, 2021
(86) PCT No.: PCT/CA2021/050771

§ 371 Date: Dec. 2, 2022

Related U.S. Application Data
(60) Provisional application No. 63/034,601, filed on Jun. 4, 2020.

(51) Int. Cl. C07D 307/91; C07D 209/88; A61K 45/06

(52) U.S. Cl. CPC C07D 307/91; A61K 45/06; C07D 209/88

(57) ABSTRACT

Disclosed herein are novel rigid cannabidiol (CBD) analogues and their formulations that modulate cannabinoid receptors, method(s) of preparation, methods of modulating cannabinoid receptors, and methods of treating various conditions related to the modulation of cannabinoid receptors, such as, pain and inflammation, cancer, glaucoma, neurodegenerative disorders, multiple sclerosis, renal fibrosis, fibrotic disorders, addiction, anxiety, insomnia, motor function disorders and gastrointestinal and metabolic disorders.

### FIELD OF THE INVENTION

The present invention relates to the field of medicinal chemistry. Specifically, it relates to methods of treating human or animal subjects in need of treatment, utilizing rigid CBD analogues as selective and specific cannabinoid receptor ligands.

### BACKGROUND

The endocannabinoid system mediates many important physiological functions including neuroplasticity and learning, emotion and motivation, appetite, and GI motility as well as immunomodulation. There are at least two types of G-protein coupled cannabinoid receptors that have been isolated and fully characterized in mammals: a) CB1: located centrally and peripherally and involved mainly in neurotransmitters homeostasis; b) CB2: located peripherally and linked with the immune system. These receptors represent promising therapeutic targets for various conditions including chronic pain, inflammation, neurodegenerative disorders, epilepsy, addiction, insomnia, cancer, obesity, and anorexia. Designing specific cannabinoid ligands to manage these conditions has received increased interest in recent years.

The cannabinoid receptors can be modulated by a heteromorphic group of compounds so-called cannabinoids. They can be classified into three main groups: a) endogenous or endocannabinoids e.g. arachidonoylethanolamide; b) natural or phytocannabinoids, which are the active constituents of Cannabis species (e.g. delta-9-tetrahydrocannabinol (THC), cannabinol (CBN), cannabielsoin (CBE), cannabifuran, and cannabidiol (CBD); c) synthetic (e.g. nabilone) (see Table 1).

TABLE 1

Representative examples of cannabinoids

| | | |
|---|---|---|
| Cannabinoids class | Examples | |
| A. Endogenous | 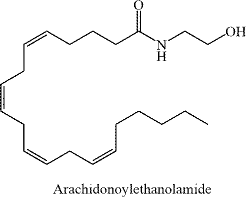 | |
| B. Natural | 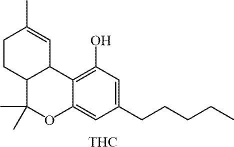 | 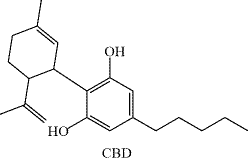 |
| C. Synthetic | 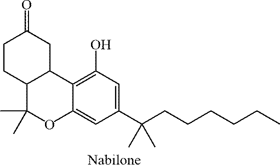 | |

Cannabidiol (CBD) can be described as a flexible resorcinol derivative, when compared to the more rigid THC phenolic structure. This may explain the greater affinity and potency of THC, where THC is a partial agonist on both CB1 & CB2 (Ki CB1/CB2 = 40.7/36.4 nM) and CBD is a partial antagonist on CB1 and weak inverse agonist on CB2 (Ki CB1/CB2 = 4350/2860 nM). Nevertheless, low selectivity is reported for both THC and CBD. In addition, CBD can be cyclized to other cannabinoids including THC, cannabielsoin (CBE) and cannabifuran (CBF) under a variety of conditions—light, pH, heat, and enzymes. This finding may explain the more psychotropic action of smoked cannabis, which may contains traces of CBE derivatives, obtained by heating CBD. When compared to THC and CBD, these CBE derivatives could be a more potent ligand to CB1 and CB2.

The clinical utility of THC and CBD and other synthetic analogues is well documented in many conditions. Sativex®, by GW Pharmaceuticals, is a buccal spray of THC and CBD in a 1:1 mixture and has been approved in many countries as an adjunctive treatment of neuropathic pain and spasticity associated with multiple sclerosis in adults. Cesamet® (nabilone), by Bausch Health Co, is a synthetic cannabinoid for oral administration as an antiemetic through a CB1 receptor mediated interaction. Yet, biological and pharmacological data on CBE and its derivatives are not known. This may be explained by its isolation in only trace amounts.

Despite their clinical potential, natural cannabinoids (phytocannabinoids) extracted from C. Sativa are highly lipophilic (log P values of 6-7), sparingly soluble in water (solubility = 2-10 µg/mL at 23° C.), chemically unstable (particularly in solution via light, temperature, and auto-oxidation), and gummy in nature with erratic absorption, a delayed onset, extensive first-pass metabolism, high protein binding, large volume of distribution and low systemic bioavailability after oral administration leading to unpredictable time course of action and long half-life (t_{½)}. In addition, the clinical benefits of smoked herb are short and associated with mucosal damage, serious adverse effects, and exposure to carcinogenic by-products. Furthermore, THC can cross blood brain barrier (BBB) and activate central CB1 producing unwanted psychotropic effect. In attempt to overcome these limitations, a variety of formulations and drug delivery approaches have been developed including co-solvency, complexation, surfactant and carrier-assisted methods. Thus far, with a limited success.

On the other hand, several synthetic analogues have been reported and widely used to modulate CB1 and CB2. Their selectivity, molecular mode of action and pharmacokinetic properties have been broadly characterized and optimized.

WO 2006/129318 A2 of Pharm-Mos Corp. discloses benzofuran derivatives, their compositions and uses thereof, but only compounds where the benzofuran is fused to an alicyclic ring, while excluding phenyl rings. These benzofuran derivatives may harbor substituted or unsubstituted alkyl chain, cycloalkyl or heterocyclic rings at C3. Moreover, certain compounds share some pharmacological properties and therapeutic indications with cannabinoids.

WO 2000/008007 A2, WO 2000/007579 A2 and WO 2003/045375 A1 of Bayer disclose the synthesis of related cyclopenta[b]benzofuran derivatives and use thereof for the treatment of nuclear factor λB-tdependent diseases. At the ring junction in formula I between the cyclopentane and the benzofuran ring, these compounds are always substituted with hydroxy and phenyl groups with a cis configuration. Moreover, these cyclopentabenzofuran derivatives harbor an additional phenyl adjacent to the ring junction at position C9 (formula I).

DE 199 34 952 of Novartis also refers to cyclopentabenzofuran derivatives, but discloses only compounds wherein ring A (formula I), according to the nomenclature adopted in the present application, is preferably substituted by methoxy groups. As in the case of the Bayer applications, the compounds of DE 199 34 952 have a fixed phenyl group at the ring junction between the cyclopentane and the benzofuran ring. Moreover, these specific compounds are attributed agro-chemical use as acaricides and insecticides, and are not contemplated as medicaments.

WO2020031179A1 and several academic publications (e.g., J. Org. Chem. 2020, 85, 2704-2715; Chem. - Asian J. 2019, 14, 3749-3762; Org. Lett. 2018, 20, 381-384; J. Org. Chem. 1992, 57, 3627-3631; Org. Lett. 2007, 9, 861-863; Gazetta Chimica Italiana, 1973, 103,127-139) disclose several synthetic routes for the preparation of cannabinoid compounds, including dibenzofuran derivatives, CBE, CBF, indoles (e.g. WIN 55, 212-2), pinene derivatives (e.g. HU-308), pyrazoles (e.g. SR 141716A). These routes usually require multiple and complex steps resulting in higher cost of production and, in many cases, the compounds are isolated in low yields or as isomers with no biological data or insufficient information about their activities.

The crystal structure of CB1 with agonist and antagonist and CB2 with antagonist have been recently published and could inform rational and more selective structure-based drug design. When compared to other approaches, this strategy may offer optimal drug candidates with improved pharmacokinetics and pharmacodynamics properties. Moreover, the designed products may be more specific and selective with tailored actions and fewer adverse effects. However, this strategy may involve tedious organic syntheses, biochemical reactions, and purification work. In addition, final products may contain impurities and/or require activation within the body.

To minimize the limitations in the prior art, there exists a demand for new drug candidates to modulate the cannabinoid receptors with optimized physicochemical, pharmacokinetic and pharmacodynamic properties for specific clinical applications.

### SUMMARY OF THE INVENTION

The compounds, according to the present invention, have a rigid pharmacophore to selectively modulate CB1/CB2 receptors. The compounds contain the pharmacophore requirements of cannabinoids ligands according to the general formula presented in formula I, thereby modulating the cannabinoid receptor, which may afford a superior and a potent approach to manage pain and inflammation and related disorders. The compounds can be considered as semi-synthetic analogues of CBD with improved chemical stability, optimized solubility, PK and PD properties.

One embodiment of the present invention is a compound comprising a central core of a fused tricyclic system, preferably substituted at C3, C5, C6 and C9 positions. Preferably, at least one of the rings (A, B and C) is an aromatic ring and one of the substituents (R1, R2, R3, R4) is an aliphatic side chain. Also included are physiologically acceptable isomers, salts, derivatives or pro-drugs of the compounds and mixture thereof.

Another embodiment relates to an optimized and versatile method to convert CBD to the target analogues, including synthetic, semisynthetic, microbial, enzymatic, synthetic biology and genetic manipulation of Cannabis sp.

The compounds can act as ligands for CB1 or CB2, or both, or exert their actions through non-receptor mediated mechanism. They can also modulate other targets and receptors, including COX enzymes, fatty acid amide hydrolase (FAAH), transient receptor potential cation channel subfamily V (TrpV), peroxisome proliferator-activated receptors, putative abnormal-cannabidiol receptor, ion channels, ligand gated ion channels and other G- protein coupled receptors.

In another embodiment, the disclosed compounds can act through agonistic or antagonistic modulation of the biological targets. In addition, the pharmacological actions of these compounds can be receptor or non-receptor mediated mechanisms.

In another embodiment, the compounds can act on both peripheral and central tissues or restricted to any of them.

In another embodiment, the compounds are peripherally restricted that lack the central psychoactive properties of THC.

In another embodiment, the compounds can be used to manage several conditions including pain and inflammation, cancer, glaucoma, neurodegenerative disorders, multiple sclerosis, renal fibrosis, fibrotic disorders, addiction, anxiety, insomnia, motor function disorders and gastrointestinal and metabolic disorders and others.

In another embodiment, the compounds can be used for both human and animal applications.

In another embodiment, the compounds can be formulated for transdermal, transmucosal (nose, oral, GIT), ophthalmic or parenteral delivery systems.

In another embodiment, the synthesized compounds include all possible isomers (stereo or structural) either as individual active compounds, salts, prodrugs or mixture thereof.

In another embodiment, the formulations can include other synergistic ingredients, including other cannabinoids, phytochemicals, analgesics and anti-inflammatory.

When compared to other ligands, the present invention discloses cannabinoids with improved PK and PD profile including better stability, solubility and taste, efficient absorption and distribution, higher affinity, selectivity, and potency which may provide effective pain control and anti-inflammatory effects.

### BRIEF DESCRIPTION OF THE DRAWINGS

In order that the invention may be more clearly understood, a preferred embodiment thereof will now be described in detail by way of example, with reference to the accompanying drawings, in which:

FIG. 1 is a molecular docking schematic view of a compound, according to the present invention, compared to a potent agonist. Example compound EX-1 (green) selectively lies in the agonist region and is aligned with the potent crystallized agonist (red) in CB1 binding site (PDB code = 5TGZ). EX-1 is away from TRP 356 and interacts with the other toggle switch PHE200 to further enhance the induced fit initiated by TRP356.

FIG. 2 is a molecular docking schematic view of the compound of FIG. 1, compared with THC. EX-1 (green) showed better conformation than THC (red) in CB1 binding site (PDB code = 5XR8). EX-1 is better oriented in the agonist site and showed conformations that keep its pentyl side chain away from TRP356.

FIG. 3 is a molecular docking schematic view of the compound of FIG. 1, compared with an antagonist (red) in CB2 (PDB code = 5ZTY). EX-1 (green) selectively occupied the agonist binding region and away from TRP256.

### DETAILED DESCRIPTION OF THE INVENTION

This disclosure relates to compounds that can modulate cannabinoids receptors, to methods of their synthesis, to pharmaceutical formulations of these compounds, to methods of modulating CB1 and CB2 and to methods of treating pain, inflammation, neurodegenerative disorders, cancer, renal fibrosis, epilepsy and other motor dysfunction, obesity and other metabolic disorder, addiction, sleep disorders, anxiety, multiple sclerosis, anorexia and others.

The term “target compounds” in the present disclosure include any, and all possible isomers, including isomers, stereoisomers, enantiomers, diastereomers, tautomers, salts, and pro-drugs thereof. They can include derivatives and analogues of a rigid pharmacophore with the general formula I. Preferably they are derivatives and analogues of a fused tricyclic system. More preferably, they are derivatives and analogues of a carbazole heterocycle. Examples of the target compounds include the compounds of formula I:

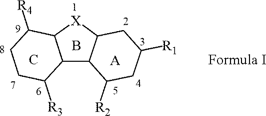

| | | |
|---|---|---|
| Rings A and C | Ring B | Arms (R₁-R₄) |
| alicyclic, heterocyclic, aromatic and heteroaromatic rings of any size, fused or non- fused | small size ring (3-5 atoms); X = C, S, O, or N | Aromatic, heteroaromatic, aliphatic chain, straight or branched, cyclic or acyclic, with/without heteroatoms, alkene, alkyne, sulfonyl, sulfonamides, basic, acidic functional group or their isosteres |

The term “a fused tricyclic system” relates to rings A, B and C with a fused relationship. The terms “substituents” relates to substituents R₁-R₄ on the peripheral carbon skeleton at C3, C5, C6 and C9.

Unless otherwise specifically defined, the term “Rings A and C” refers to alicyclic (e.g. cyclohexane), heterocyclic (e.g. tetrahydropyridine), aromatic (e.g. benzene) or heteroaromatic (e.g. pyridine) rings of any size. Preferably, of 3 to 8 atoms. Preferably, ring A is an aromatic or heteroaromatic with six atoms size, while ring C is an alicyclic ring, optionally saturated or unsaturated, optionally aromatic or heteroaromatic. The rings A and C could be fused (e.g. naphthalene or indole) or non-fused systems (e.g. benzene or pyrrole). Preferably, rings A and C are not fused systems. The rings A and C may be optionally substituted by one or more substituents at any point of attachment. Preferably, rings A and C are substituted. Preferably, the substituents on ring A have a 1,3-relationship and a 1,4-relationship on ring C. The substituents can themselves be optionally substituted and modified to include hydrogen, hydroxyl, hydroxymethyl, halogens (F, Cl, Br, I), methoxy, ethoxy, nitrile, amino, nitro and halogenated or cyanated alkyl groups (e.g. CF₃, -CH2CN). The rings A and C may optionally contain a hetero atom including, N, O, S. Preferably, ring A and C may contain nitrogen. When rings A or C contain a basic nitrogen, the target compounds are protonated at physiological pH, and may show decreased ability to cross the blood brain barrier. As a result, they exert their actions peripherally without any psychotropic effects.

In order to enforce rigidity of the target molecules, ring B is a small size ring of 3-5 atoms of any nature, including aliphatic, aromatic, heteroaromatic, saturated or unsaturated. Preferably, ring B is composed of five atoms with at least one carbon-carbon single bond. At least one atom (atom X) of ring B is a heteroatom including S, O and N. Preferably, ring B contain a basic NH to act as a H-bond donor group. This atom (X) is in a meta relation to substituents R₁ and R₂.

The substituents R₁-R₄ could be of any nature including aromatic, aliphatic, saturated or unsaturated, linear or branched chain, cyclic or acyclic hydrocarbon, halogen or hetero based functionality, saturated or unsaturated heterocyclic ring, as defined for rings A-C. Preferably, C3 substituent (R₁) is an aliphatic chain, optionally substituted with a hydrogen donor or acceptor group. Examples include, methyl (“Me”), ethyl (“Et”), propyl, isopropyl, cyclopropyl, n-butyl, t-butyl, sec-butyl, isobutyl, cyclobutyl, pentyl, cyclopentyl, hexyl, isohexyl, cyclohexyl, heptyl, cycloheptyl, 4,4-dimethylpentyl, 1,1-dimethylpentyl, 1,2-dimethylheptyl, octyl, 2,2,4-trimethylpentyl, nonyl, decyl, undecyl, dodecyl, adamantly and their cyclic and alicyclic analogues and the like. C5 substituent (R₂) is a hydrogen donor group (e.g. OH, SH, NH, NH₂, SO₂NH₂). C6 substituent (R₃) is an aromatic or heteroaromatic or unsaturated ring, optionally substituted or unsubstituted. C9 substituent (R4) is a small alkyl chain (e.g. methyl, ethyl, propyl), optionally substituted with a hydrogen donor group (e.g. OH, SH, NH, NH₂, SO₂NH₂). The substituents R₁-R₄ may be optionally substituted by a hetero atom or a halogen, including F, Cl, Br, I, O, N, and S and may contain aromatic or heteroaromatic rings. The substituents R₁-R₄ may contain other functional groups, including alkene, alkyne, alcohol, carbonyl, amine, nitro, ether, acid, sulfonyl, sulfonamides, amide, ester, cyano and a substituted or unsubstituted aryl group.

In a preferred embodiment, the selective cannabinoid ligands of the present invention are represented by the following examples (EX-1 to EX-18):

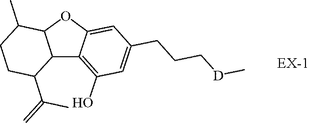

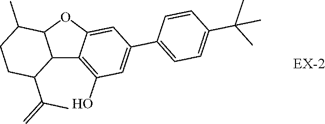 [SMILES: C=C(C)C1CCC(C)C2Oc3cc(-c4ccc(C(C)(C)C)cc4)cc(O)c3C12]

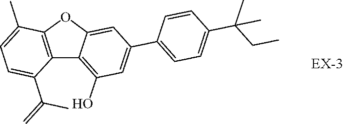 [SMILES: C=C(C)c1ccc(C)c2oc3cc(-c4ccc(C(C)(C)CC)cc4)cc(O)c3c12]

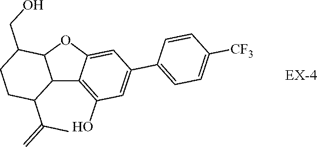 [SMILES: C=C(C)C1CCC(CO)C2Oc3cc(-c4ccc(C(F)(F)F)cc4)cc(O)c3C12]

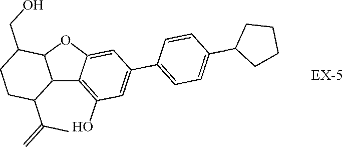 [SMILES: C=C(C)C1CCC(CO)C2Oc3cc(-c4ccc(C5CCCC5)cc4)cc(O)c3C12]

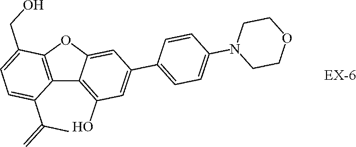 [SMILES: C=C(C)c1ccc(CO)c2oc3cc(-c4ccc(N5CCOCC5)cc4)cc(O)c3c12]

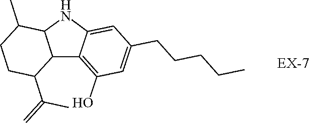 [SMILES: C=C(C)C1CCC(C)C2Nc3cc(CCCCC)cc(O)c3C12]

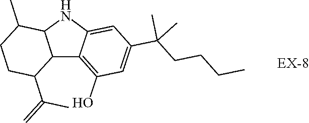 [SMILES: C=C(C)C1CCC(C)C2Nc3cc(C(C)(C)CCCC)cc(O)c3C12]

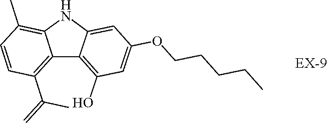 [SMILES: C=C(C)c1ccc(C)c2[nH]c3cc(OCCCCC)cc(O)c3c12]

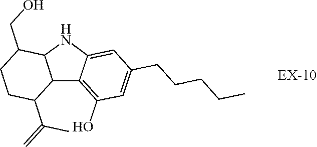 [SMILES: C=C(C)C1CCC(CO)C2Nc3cc(CCCCC)cc(O)c3C12]

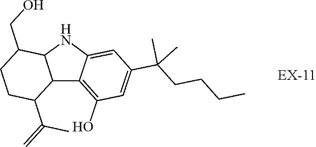 [SMILES: C=C(C)C1CCC(CO)C2Nc3cc(C(C)(C)CCCC)cc(O)c3C12]

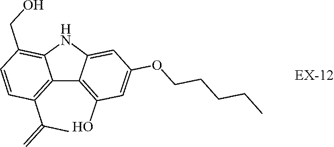 [SMILES: C=C(C)c1ccc(CO)c2[nH]c3cc(OCCCCC)cc(O)c3c12]

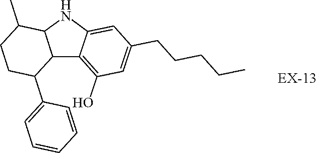 [SMILES: CCCCCc1cc(O)c2c(c1)NC1C(C)CCC(c3ccccc3)C21]

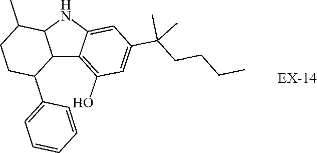 [SMILES: CCCCC(C)(C)c1cc(O)c2c(c1)NC1C(C)CCC(c3ccccc3)C21]

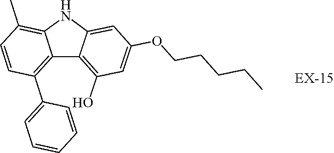 [SMILES: CCCCCOc1cc(O)c2c(c1)[nH]c1c(C)ccc(-c3ccccc3)c12]

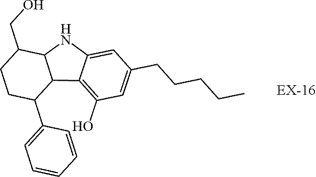 [SMILES: CCCCCc1cc(O)c2c(c1)NC1C(CO)CCC(c3ccccc3)C21]

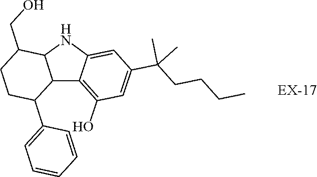 [SMILES: CCCCC(C)(C)c1cc(O)c2c(c1)NC1C(CO)CCC(c3ccccc3)C21]

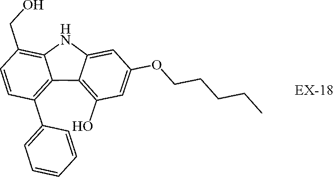 [SMILES: CCCCCOc1cc(O)c2c(c1)[nH]c1c(CO)ccc(-c3ccccc3)c12]

The compounds may be prepared according to the reaction below, where modification of the reaction can produce other derivatives and analogues. This reaction is presented as an example; however, other possible routes will become apparent to those skilled in the art.

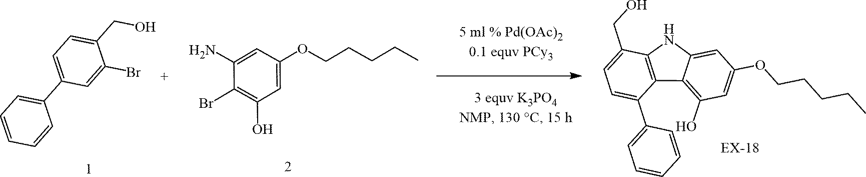

The term “pharmaceutical formulation”, as used herein, refers to a mixture of one or more of the compounds described herein, or pharmaceutically acceptable salts thereof, or other synergistic compounds along with other physiologically acceptable carriers and excipients. The purpose of a pharmaceutical formulation (e.g. solid or liquid dosage forms) is to facilitate administration of a compound to a subject animal or human. Preferably, the formulation is a solid dosage form for oral and oromucosal applications.

The formulation may contain synergistic ingredients, in addition to active ingredients, which may include: delta-9-tetrahydrocannabinol (THC); delta-8-tetrahydrocannabinol (THC); cannabidiol (CBD); cannabinodiol (CBND); cannabinol (CBN); cannabigerol (CBG); cannabichromene (CBC); cannabicyclol (CBL); canabivarol (CBV); tetrahydrocannabivarin (THCV); cannabidivarin (CBDV); cannabichromevarin (CBCV); cannabigerol monoethyl ether (CBGM); cannabielsoin (CBE); cannabitriol (CBT); Boswellia sp. extracts, including Boswellia carterii and Boswellia serrata; ginger; capsaicin; camphor; polyphenols, including quercetin, ellagic acid, curcumin, and resveratrol; phytosterols; carbohydrates, including mannose-6-phosphate; essential oils, including thymol and carvacrol; terpenoids, including squalene, lycopene, p-cymene and linalool; or mixtures or combinations thereof. Preferably, the formulation contains only one active ingredient, being the target compound, without any synergistic ingredients.

The term “salt” is intended to include salts derived from inorganic (e.g. hydrochloric) or organic acids (e.g. tartaric acid).

The term “pro-drugs” is intended to include derivatives of the target compounds that may require activation within the human body (e.g. carbohydrate and ester derivatives).

Certain exemplary compounds, according to the present invention, can be delivered by oromucosal, nasal, oral, ophthalmic, transdermal and parenteral routes. They can be used for the treatment of inflammation and pain and other related conditions. They have an improved pharmacokinetic/pharmacodynamic profile, compared to some other related analogues. This is useful in the treatment of inflammation, pain, and related conditions to quickly alleviate the symptoms and provide long-lasting relief to the patient.

The term “subject” in the present disclosure refers to human patients but may include animals.

### Example 1: Preparation of EX-18

A solution of [Pd(OAc)₂] (11.2 mg, 0.05 mmol, 5.0 mol%), PCy₃ (28.9 mg, 0.10 mmol, 10 mol%), K₃PO₄ (636.8 mg, 3.00 mmol), aryl halide 1 (263.1 mg, 1.00 mmol) and aniline derivative 2 (329 mg, 1.20 mmol) in dry NMP (10.0 mL) was stirred for 15 h at 130° C. under N₂. Et₂O (25 mL) and H₂O (25 mL) were added to the mixture at r.t. and the separated aqueous phase was extracted with Et₂O (2 × 70 mL). The combined organic layers were washed with brine (50 mL), dried (sodium sulfate), and concentrated in vacuo. The residue was purified by column chromatography (silica gel, pentane) to give EX-18.

### Example 2: Molecular Docking Studies of EX-1 Compared to Potent Ligands

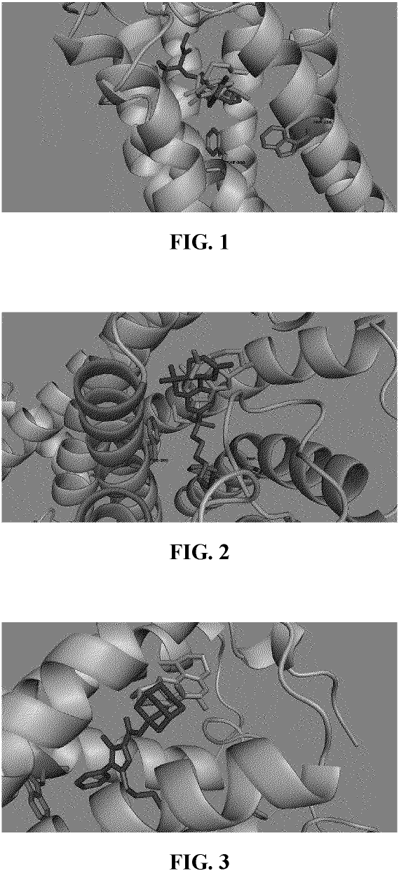

As shown in FIG. 1, EX-1 selectively lies in the agonist region and is aligned with the potent crystallized agonist. EX-1 is away from TRP356 and interacts with the other toggle switch PHE200 to further enhance the induced fit initiated by TRP356.

As shown in FIG. 2, EX-1 showed better conformation than THC in the CB1 binding site. EX-1 is better oriented in the agonist site and showed conformations that keep its pentyl side chain away from TRP356.

As shown in FIG. 3, compared to an antagonist shown in red, EX-1 selectively occupied the agonist binding region and is positioned away from TRP256, which is labeled as magenta sticks.

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A compound of formula I: 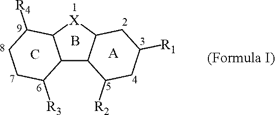
wherein: X = C, S, O, or N; and
R₁, R₂, R₃, and R₄ are independently selected from the group consisting of: CH₃, CH₂OH, OH, CCH₂CH₃, a halogen, methoxy, ethoxy, nitrile, amino, nitro, halogenated or cyanated alkyl groups, aromatic, heteroaromatic, and cyclic or acyclic aliphatic chains; and
wherein at least one of R₁, R₂, R₃, or R₄ is an aromatic, heteroaromatic, cyclic or acyclic aliphatic chain.
wherein if X = O, R₁ is a substituted or unsubstituted aromatic or heteroaromatic.

2. The compound of claim 1, wherein one or both of ring A and ring C contain a heteroatom.

3. The compound of claim 2, wherein the heteroatom of one or both of ring A and ring C is, independently, N, O, or S.

4. The compound of claim 1, wherein R₂ is a hydrogen donor group.

5. The compound of claim 4, wherein R₂ is selected from the group consisting of: OH, SH, NH, NH₂, SO₂NH₂.

6. The compound of claim 5, wherein R₃ is an aromatic, heteroaromatic, or unsaturated ring.

7. The compound of claim 6, wherein R₄ is a one to three-carbon alkyl chain.

8. The compound of claim 7, wherein the R₄ alkyl chain is substituted with a hydrogen donor group.

9. The compound of claim 8, wherein R₁ is a substituted or unsubstituted aromatic or heteroaromatic.

10. The compound of claim 9, wherein R₁ is substituted with a group selected from the group consisting of: methyl, ethyl, propyl, isopropyl, cyclopropyl, n-butyl, t-butyl, sec-butyl, isobutyl, cyclobutyl, pentyl, cyclopentyl, hexyl, isohexyl, cyclohexyl, heptyl, cycloheptyl, 4,4-dimethylpentyl, 1,1-dimethylpentyl, 1,2-dimethylheptyl, octyl, 2,2,4-trimethylpentyl, nonyl, decyl, undecyl, dodecyl, adamantly and their cyclic and alicyclic analogues.

11. The compound of claim 10, wherein one or more of R₁-R₄ are substituted by a heteroatom or a halogen.

12. The compound of claim 11, wherein one or more of R₁-R₄ are selected from the group consisting of: an alkene, alkyne, alcohol, carbonyl, amine, nitro, ether, acid, sulfonyl, sulfonamide, amide, ester, cyano, and a substituted or unsubstituted aryl group.

13. The compound of claim 1, wherein the compound is.

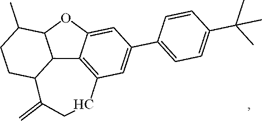

14. The compound of claim 1, wherein the compound is.

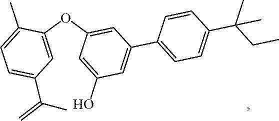

15. The compound of claim 1, wherein the compound is.

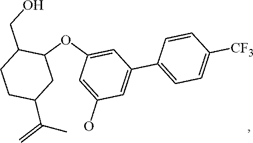

16. The compound of claim 1, wherein the compound is.

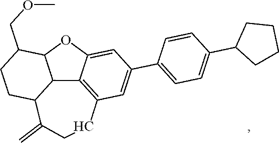

17. The compound of claim 1, wherein the compound is.

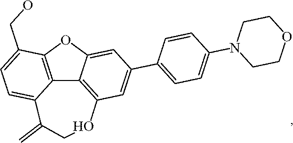

18. The compound of claim 1, wherein the compound is.

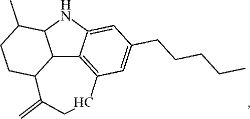

19. The compound of claim 1, wherein the compound is.

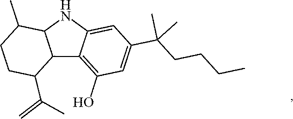

20. The compound of claim 1, wherein the compound is.

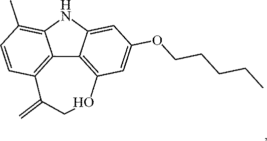

21. The compound of claim 1, wherein the compound is.

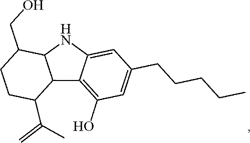

22. The compound of claim 1, wherein the compound is.

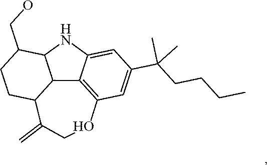

23. The compound of claim 1, wherein the compound is.

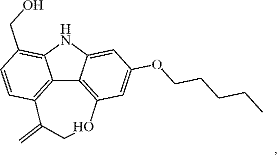

24. The compound of claim 1, wherein the compound is.

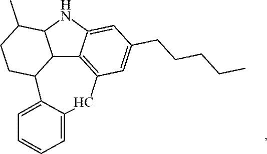

25. The compound of claim 1, wherein the compound is.

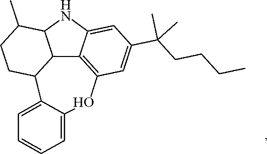

26. The compound of claim 1, wherein the compound is.

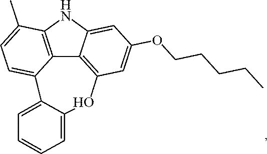

27. The compound of claim 1, wherein the compound is.

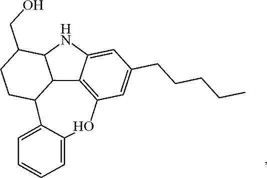

28. The compound of claim 1, wherein the compound is.

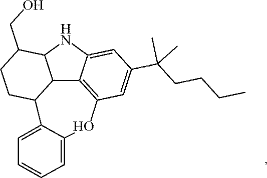

29. The compound of claim 1, wherein the compound is.

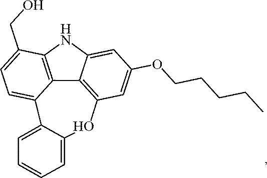

30. A compound comprising a tricyclic heterocycle having a ring A and a ring C fused on either side of a ring B, wherein ring A and ring C are individually selected from the group consisting of: alicyclic, heterocyclic, aromatic and heteroaromatic rings, and wherein ring B is a three to five atom ring with a C, S, O, or N atom;
wherein each of ring A and ring C have at least two side groups individually selected from the group consisting of: CH₃, CH₂OH, OH, CCH₂CH₃, a halogen, methoxy, ethoxy, nitrile, amino, nitro, halogenated or cyanated alkyl groups, substituted or unsubstituted aromatic, heteroaromatic, and cyclic or acyclic aliphatic chains;
wherein at least one of ring A and ring C have a side group selected from the group consisting of: a substituted or unsubstituted aromatic, heteroaromatic, and cyclic or acyclic aliphatic chain; and
wherein ring C has an O atom, at least one of the side groups on ring A or ring C is a substituted or unsubstituted aromatic or heteroaromatic group.

31. The compound of claim 30, wherein each of ring A and ring C is a three to eight atom ring.

32. The compound of claim 31, wherein at least one of ring A and ring C is an aromatic, heteroaromatic, or unsaturated ring.

33. The compound of claim 32, wherein the two side groups on ring A have a 1,3-relationship and the two side groups on ring C have a 1,4-relationship.

34. The compound of claim 33, wherein one or more of ring A or C contains a basic nitrogen.

35. The compound of claim 33, wherein at least one of ring A and ring C has a hydrogen donor side group.

36. The compound of claim 35, wherein the hydrogen donor side group is selected from the group consisting of: OH, SH, NH, NH₂, SO₂NH₂.

37. The compound of claim 36, wherein at least one of ring A and ring C has a one to three-carbon cyclic or acyclic alkyl chain side group.

38. The compound of claim 37, wherein the alkyl chain side group is substituted with a hydrogen donor group.

39. The compound of claim 38, wherein at least one of ring A and ring C has a cyclic or acyclic aliphatic side chain.

40. The compound of claim 39, wherein the aliphatic side chain is selected from the group consisting of: methyl, ethyl, propyl, isopropyl, cyclopropyl, n-butyl, t-butyl, sec-butyl, isobutyl, cyclobutyl, pentyl, cyclopentyl, hexyl, isohexyl, cyclohexyl, heptyl, cycloheptyl, 4,4-dimethylpentyl, 1,1-dimethylpentyl, 1,2-dimethylheptyl, octyl, 2,2,4-trimethylpentyl, nonyl, decyl, undecyl, dodecyl, adamantly and their cyclic and alicyclic analogues.

41. The compound of claim 40, wherein one or more of the side groups on ring A and ring C are substituted by a heteroatom or a halogen.

42. The compound of claim 41, wherein one or more of the side groups on ring A and ring C are selected from the group consisting of: an alkene, alkyne, alcohol, carbonyl, amine, nitro, ether, acid, sulfonyl, sulfonamide, amide, ester, cyano, and a substituted or unsubstituted aryl group.

43. The use of a compound of claim 1 to treat one or more conditions selected from pain and inflammation, cancer, glaucoma, neurodegenerative disorders, multiple sclerosis, renal fibrosis, fibrotic disorders, addiction, anxiety, insomnia, motor function disorders and gastrointestinal and metabolic disorders.

44. A pharmaceutical formulation, comprising a compound of claim 1 or physiologically acceptable isomers, salts, derivatives, or pro-drugs and mixtures thereof.

45. The pharmaceutical formulation of claim 44, comprising a synergistic compound selected from the group consisting of: delta-9-tetrahydrocannabinol (THC); delta-8-tetrahydrocannabinol (THC); cannabidiol (CBD); cannabinodiol (CBND); cannabinol (CBN); cannabigerol (CBG); cannabichromene (CBC); cannabicyclol (CBL); canabivarol (CBV); tetrahydrocannabivarin (THCV); cannabidivarin (CBDV); cannabichromevarin (CBCV); cannabigerol monoethyl ether (CBGM); cannabielsoin (CBE); cannabitriol (CBT); Boswellia sp . extracts, including Boswellia carterii and Boswellia serrata; ginger; capsaicin; camphor; polyphenols, including quercetin, ellagic acid, curcumin, and resveratrol; phytosterols; carbohydrates, including mannose-6-phosphate; essential oils, including thymol and carvacrol; terpenoids, including squalene, lycopene, p-cymene and linalool.
